(19) United States
(12) Patent Application Publication
(10) Pub. No.: US 2024/0424463 A1
(43) Pub. Date: Dec. 26, 2024

(54) COOLED REACTOR FOR PERFORMING EXOTHERMIC EQUILIBRIUM REACTIONS

(71) Applicant: L'Air Liquide, Societe Anonyme pour l'Etude et l’Exploitation des Procedes Georges Claude, Paris (FR)

(72) Inventors: Frank CASTILLO-WELTER, Frankfurt am Main (DE); Nga Thi Quynh DO, Erlangen (DE); Stephane HAAG, Frankfurt am Main (DE)

(73) Assignee: L'Air Liquide, Societe Anonyme pour l'Etude et l’Exploitation des Procedes Georges Claude, Paris (FR)

(21) Appl. No.: 18/751,124

(22) Filed: Jun. 21, 2024

(30) Foreign Application Priority Data
Jun. 23, 2023 (EP) EP 23181210

(51) Int. Cl. B01J 8/06; B01J 8/02; C01C 1/04; C07C 29/152

(52) U.S. Cl. CPC B01J 8/067; B01J 8/0278; B01J 8/0285; B01J 8/065; C01C 1/0417; C07C 29/152; B01J 2208/00194; B01J 2208/00309; B01J 2208/022

(57) ABSTRACT

What is proposed is a reactor for performing exothermic equilibrium reactions, in particular for performing methanol synthesis and/or ammonia synthesis, by heterogeneously catalysed reaction of the corresponding reactant gases which makes it possible to overcome the establishment of the reaction equilibrium in the reactor. To this end, according to the invention, the coolant temperature is influenced and thus optimized along the longitudinal coordinate of the reactor through subcooling of the coolant.

### CROSS-REFERENCE TO RELATED APPLICATIONS

This application claims the benefit of priority under 35 U.S.C. § 119 (a) and (b) to EP patent application No. EP 23181210, filed Jun. 23, 2023, the entire contents of which are incorporated herein by reference.

### BACKGROUND

### Field of the Invention

The present invention relates to a reactor for performing exothermic equilibrium reactions where a gaseous input mixture is at least partially reacted to afford a product mixture over a solid catalyst. Example applications include performing methanol synthesis by reaction of synthesis gas comprising hydrogen and carbon oxides and performing ammonia synthesis by reaction of input gas comprising hydrogen and nitrogen, in each case as a heterogeneously catalysed reaction over solid catalysts.

### Related Art

Reactors for performing exothermic equilibrium reactions have long been known to those skilled in the art. An industrially particularly important reaction of this type is, for example, methanol synthesis by heterogeneously catalysed reaction of synthesis gas, i.e. mixtures of hydrogen and carbon oxides. Ullmann's Encyclopedia of Industrial Chemistry, Sixth Edition, 1998 Electronic Release, chapter “Methanol”, subchapter 5.2 “Synthesis” describes various basic processes for producing methanol by catalytic conversion of synthesis gas comprising hydrogen and carbon oxides in which such reactors are employed.

A further exothermic equilibrium reaction of particular industrial importance is ammonia synthesis, which is explained in detail in Ullmann's Encyclopedia of Industrial Chemistry, Sixth Edition, 1998 Electronic Release, chapter “Ammonia”, subchapter 4 “Production”.

A modern two-stage process for producing methanol is disclosed in European patent specification EP 0 790 226 B1 for example. The methanol is produced in a circular process wherein a mixture of fresh and partially reacted synthesis gas is supplied initially to a water-cooled reactor and then to a gas-cooled reactor, in each of which the synthesis gas is converted into methanol over a copper-based catalyst. The methanol produced in the process is separated from the synthesis gas to be recycled which is then passed through the gas-cooled reactor in countercurrent as coolant and preheated to a temperature of 220° C. to 280° C. before it is introduced into the first synthesis reactor. A portion of the synthesis gas to be recycled is removed from the process as a purge stream to prevent inert components from accumulating in the synthesis circuit. This measure is also taught in German laid-open specification DE 2934332 A1 and European patent application EP 1016643 A1.

Unconverted methane from synthesis gas production is considered an inert component in the context of methanol synthesis and also ammonia synthesis since this compound does not undergo further conversion under the conditions of methanol or ammonia synthesis. The same applies to argon which passes into synthesis gas production via feed streams.

The water-cooled reactor stage typically achieves the main conversion of the synthesis gas (CO, CO₂, H₂) and removes the largest portion of the reaction heat while the gas-cooled stage converts a nevertheless considerable portion of the synthesis gas under milder conditions.

The water-cooled reactor (WCR) is typically a shell-and-tube reactor comprising corresponding tube end plates, wherein the catalyst is filled into the tubes while cooling is effected by means of boiling water/steam generation on the shell side around the tubes (interior space). In the gas-cooled reactor (GCR) cooling is effected with the input gas which is passed through the tubes and is heated on the way to the first reaction stage (WCR) while the catalyst is filled around the tubes and the reaction takes place on the shell side of the GCR. In terms of their nominal width the reaction stages are connected with large or very large pipe conduits; depending on plant capacity pipe diameters of up to 1 m are possible. This is attributable above all to the large gas amounts which are recycled to the second stage (recycle gas) and admixed with the fresh gas, i.e. fresh synthesis gas from gas production. After preheating in the GCR the resulting gas mixture of recycle gas and fresh gas is supplied to the first reaction stage (WCR). The recycle gas amount is typically markedly greater than the fresh gas amount and depends on the achieved conversion in the reactor section. The recycle ratio RR (RR=R/F) of recycle gas amount (R) to fresh gas amount (F) is often above 2 and in many cases even above 3.5. The lower the per-pass conversion of synthesis gas by the reactor section, the higher the recycle ratio RR required to achieve sufficient yield.

This leads to a corresponding increase in the circulating gas quantity which increases the space velocity of the reactors and requires greater nominal pipe widths of the connecting pipe conduits and also results in an increased demand for compression energy (higher flow rate and pressure drop).

A current process for ammonia synthesis is described for example in patent publication WO 2002/038499 A1. Compared to the synthesis gas used for methanol synthesis it is important in the case of synthesis gas for ammonia synthesis to completely eliminate the proportion of carbon oxides, so that hydrogen passes into the ammonia synthesis as the sole remaining synthesis gas constituent. This is effected initially through conversion of the carbon monoxide present in the synthesis gas (CO conversion), subsequent carbon dioxide removal by means of a sorption process and finally by means of cryogenic gas fractionation.

It is a disadvantage of the aforementioned synthesis reactions that as exothermic equilibrium reactions they are limited by the reaction equilibrium. It would therefore be desirable to specify reactors and processes for their operation which make it possible to overcome these limitations.

### SUMMARY OF THE INVENTION

It is accordingly an object of the present invention to specify a reactor and a process for operation thereof which does not have the described disadvantages of the reactors known from the prior art and which especially provides a high conversion for the target products of the exothermic equilibrium reaction per reactor pass and provides the opportunity to influence and thus optimize the reaction conditions along the longitudinal coordinate of the reactor which, for example in the case of methanol synthesis, leads to a reduction in the recycle ratio to smaller values such as are known when using the reactors familiar from the prior art.

This object is achieved in a first aspect by a reactor having the features of claim 1 and in a further aspect by a process having the features of claim 8. Further embodiments according to further aspects of the invention are apparent from the subsidiary claims of the respective category.

Fluid connection between two regions of the reactor according to the invention is to be understood as meaning any type of connection whatsoever which makes it possible that a fluid, for example the input gas stream or the synthesis gas product stream, can flow from the one to the other of the two regions, neglecting any interposed regions or components.

Means for introducing, discharging etc. are to be understood as meaning all apparatuses, apparatus constituents, assemblies and components which make it possible for the particular fluid to leave the spatial region under consideration, for example a container. This refers in particular to pipe conduits, pumps, compressors, other conveying apparatuses and the corresponding openings in the container wall.

Catalytic activity, especially in connection with different catalytic activities when comparing two different catalysts, is to be understood as meaning the achieved degree of conversion per unit length of the catalyst bed of reactants to products. Activity is influenced by the chemical composition, doping, poisoning, available surface area etc. of the catalyst material but also by the geometry of the catalyst particles and textural parameters of the catalyst bed, for example its porosity or packing density. Due to the exothermicity of the reactions considered a high catalytic activity correlates with a high evolution of heat per unit length of the catalyst bed.

In the context of the present description a tubular reactor is to be understood as meaning a reaction apparatus comprising a tube or a multiplicity of tubes of generally circular cross section which are filled with fills of one or more catalysts, wherein the tube(s) are generally fabricated from a metallic material. The reactant stream enters the tubular reactor at one of the open tube ends, flows through it continuously and is thus converted into a product stream which is continuously withdrawn at the opposite open tube end. Axial backmixing of the material streams flowing through the tubular reactor occurs only to a small extent, if at all. Exothermic reactions therefore result in an axial temperature profile with more or less pronounced maxima (so-called hot spots) according to the heat evolution of the reaction. In the context of the present description the reaction temperature range is to be understood as meaning the temperature range encompassed by the temperature profile.

Plate reactors are to be understood as meaning a reaction apparatus where a catalyst fill/a catalyst bed of a solid particulate catalyst is arranged between two, generally parallel, plates. The sides of this arrangement that are not used for introducing reactants/discharging reaction products are generally sealed at their edges, for example by fluid-tight contact with the inner wall of the shell of the reactor or by appropriate welds.

Plate reactors are also to be understood as meaning a reaction apparatus where the catalyst bed is arranged not between two flat plates but rather between two pillow plates. A pillow plate in the context of the invention is composed of two metal sheets which are joined, preferably welded, at the edges, and over whose surface a multiplicity of additional joins, preferably spot welds, which likewise join the two plates to one another are distributed. Such plates may be produced by robots or machines in automated fashion and thus very cost-effectively. After welding, the metal sheets are expanded by hydraulic forming, generally injection of a fluid under high pressure, thus creating pillow-like channels between the metal sheets, through which a heating or cooling fluid may be passed. These heat-transfer spaces thus make it possible to both supply and remove heat energy from certain regions of the reactor through passage of heating or cooling fluids.

When using pillow plates the reaction zones may be configured such that in the reactor two pillow plates are initially arranged substantially parallel. Substantially parallel in the context of the invention is to be understood as meaning that the alignment of the pillow plates relative to one another diverges from the parallel by at most +/−20°, preferably by at most +/−10°, particularly preferably by at most +/−5°, very particularly preferably by at most +/−2°. Subsequently the free space between the pillow plates may be filled up with a fill of a solid, granular, particulate or pellet-form catalyst, wherein the lateral termination of the resulting catalyst bed is formed by nets, meshes, perforated plates, grids, fills of inert material and/or the reactor inner wall.

It is particularly preferable when are at least one, preferably two or more, further pillow plates spaced apart from one another adjoin parallel to this arrangement, thus altogether forming a plate package, and the cavities between the pillow plates are filled with catalyst fills. This makes it possible to achieve in the reaction zone a compact sandwich-like structure with an intensive cooling apparatus which extends over the length of the reaction zone. The individual catalyst fills are supplied with the reaction gas mixture in parallel. The plate packages may be aligned parallel or perpendicular to the longitudinal axis of the reactor based on the catalyst-filled cavities.

The distances between the pillow plates may be selected according to the heat evolution of the reaction to be performed: For strongly exothermic reactions this distance is made smaller than for more weakly exothermic reactions. Smaller plate distances are preferred in the first reaction zone since the greatest conversion is achieved here and the greatest heat removal must be realized here. In the case of methanol synthesis the pillow plate distances of the first reaction zone are preferably 20 to 45 mm. The distance relates to the distance from centreline to centreline, i.e. the clear distance between the plates is correspondingly smaller according to pillow plate thickness and expansion of the cavity. The distance is moreover adapted to the dimensions of the catalyst particles in order to ensure optimum heat removal and good bulk material behaviour when filling and emptying the catalyst without bridging. In the second and subsequent reaction zones, the distances are usually made larger.

Compared to tubular reactors plate reactors have the advantage that a larger cooling area per unit volume of the catalyst is generally available.

In the present description the longitudinal axis is to be understood as meaning the axis of a device or an apparatus corresponding to the direction of its greatest extent. In the context of the present description an arrangement of a device or an apparatus whose longitudinal axis is parallel to the vertical as defined by the gravitational force is to be understood as meaning an upright arrangement.

Coolants employed in the reactor cooling according to the invention are preferably media which are close to their boiling point and thus easily evaporate in the reaction temperature range at the reaction pressure. This ensures good heat removal from the catalyst bed during the exothermic reaction due to the good heat transfer on the side of the evaporating medium and allows precise temperature control via the pressure. To establish different temperature conditions in the different stages the pressure on the side of the cooling medium in the interior space is controlled. With increasing catalyst uptime the conditions may be adapted by corresponding adjustment of the pressure in the interior space, thus influencing the reaction temperature in order for example to keep the conversion correspondingly high.

Having regard to the desired reaction conditions, methanol synthesis for example may employ water or steam as the heat transfer medium. However, when using water, achieving the desired temperature range entails establishing relatively large pressure differences to cover a wide temperature range (for example 250° C.: about 40 bar, 264° C.: about 50 bar). By contrast, employing an evaporating heat transfer oil (for example Dowtherm A) in a circuit for steam generation makes it possible to operate in a very narrow pressure range while nevertheless covering a large temperature range (for example: 255° C.: 0.97 bar, 305° C.: 2.60 bar, corresponding to a temperature range of 50° C. at a pressure difference of only 1.6 bar. This makes it possible to employ a simple heat transfer oil steam drum at the corresponding plant level (about 20 to 25 m) and to utilize the geodetic feed height alone to establish the individual pressure and temperature ranges.

In the context of the present description the coolant cooling apparatus has to meet the requirement of being capable, in conjunction with the pressure control apparatus, of establishing a defined coolant temperature below the boiling point of the coolant at the pressure in the interior space (subcooling). The interior space is to be understood as meaning the free space traversable by the coolant between the outer wall of the at least one tubular reactor or the outer walls of the at least one plate reactor and the inner wall of the shell.

The conditions of an exothermic equilibrium reaction in general and specifically methanol synthesis conditions/ammonia synthesis conditions are to be understood as meaning the process conditions known per se to a person skilled in the art, in particular of temperature, pressure and residence time, as mentioned for example hereinabove and discussed in detail in the relevant literature and under which at least partial conversion, but preferably industrially relevant conversions of the reactants into the products of the respective process, takes place. The same applies to the selection of a suitable catalyst and suitable operating conditions thereof since in the context of the present invention both recited processes are operated under heterogeneous catalysis—corresponding methanol synthesis reactors and ammonia synthesis reactors are known per se to those skilled in the art and described for example in the literature recited at the outset.

The invention is based on the finding that optimal temperature management in particular in the axial direction of the reactor makes it possible to markedly improve the production rates/space-time yields along the reaction path. The temperature profile along the reaction path is considerably improved by the use of the cooling concept according to the invention, thus achieving a markedly higher conversion per pass. The reactor cooling according to the invention may be employed for catalyst-filled tubular reactors and for catalyst-filled plate reactors.

Byproduct formation during methanol synthesis is also reduced relative to the prior art when using the reactor according to the invention.

An improved temperature profile in the reactor is in principle also achievable using a catalyst layer management. This would comprise employing a less active catalyst in the region where the highest conversion (exothermicity) and thus the highest temperatures are expected and employing a more active catalyst in regions where a lower conversion is expected. However, such a catalyst layer management is relatively inflexible, since the different catalyst layers have to be selected and defined on the basis of a certain catalyst activity and a corresponding gas composition. However, the catalyst activity changes over the uptime of the synthesis plant as a result of its ongoing deactivation.

The layer management and the associated cooling of the reaction bed must be matched to one another. During the catalyst uptime and the associated catalyst deactivation the conditions change and an adapting of the reaction temperature and the accompanying cooling/cooling temperature is desirable to at least partially compensate for the deactivation and to ensure a high conversion with low byproduct formation. Reactors known from the prior art allow the cooling to be adapted only for the entire reactor; however typically not all catalyst layers undergo deactivation to the same extent over the operating time. Establishment of specific reaction conditions is therefore always a compromise.

The approach according to the invention allows the reaction conditions to be individually adapted along the longitudinal axis of the catalytic fixed bed reactor according to catalyst activity, gas composition in each stage and the potential presence of two or more layers of different catalysts (layer management) and also over the uptime. This achieves a high conversion and a low byproduct formation over the entire reactor.

The optimized temperature management also reduces the maximum temperatures (and temperature peaks, so-called hot spots) in the catalyst bed. In addition to the removal of coproduct from the reaction system, for example of water in the methanol synthesis, this also has a positive effect on catalyst uptime. It is known that high temperatures in the catalyst bed contribute to faster catalyst deactivation.

One aspect of the invention is to use a reactor setup having an evaporating cooling medium, for example water or heat transfer oil, on the cooling side of the reactor. This makes it possible to ensure very good heat transfer on the cooling side in the zones in which the cooling medium is partially evaporated. In the case of an upright arrangement of the reactor the evaporation preferably occurs in the upper portion of the reactor where the highest conversion on the reaction side occurs and a corresponding cooling is required and useful. The cooling medium is therefore introduced in liquid and subcooled form into the lower region of the cooling side of the reactor while the reaction gas enters into the reaction side from above. In principle, the cooling medium flows countercurrently to the reaction gas on the cooling side (interior space).

In one example the cooling medium is recirculated through natural circulation, i.e. through density differences due to evaporation and/or temperature differences along the cooling circuit, but a forced circulation could also be effected through the use of pumps in the cooling circuit which would pump the subcooled liquid from a steam drum as a cooling medium reservoir to the cooling side of the reactor.

To optimize reactor performance the cooling medium provided from the reservoir in a saturated state is subcooled before entry into the cooling side of the reactor. The reduced cooling temperature results in lower reaction temperatures on the process side in the reactor which is advantageous in terms of the thermodynamic equilibrium of the exothermic reaction and ultimately improves conversion rates. Lower temperatures are also advantageous in other respects (for example reduced formation of byproducts, extended service life of the catalyst).

The reactor employed may be any relevant reaction apparatuses where primarily a countercurrent flow of cooling medium and reaction gas may be realized. Examples include tubular reactors, shell-and-tube reactors and plate reactors.

The subcooling of the cooling medium may be effected using various heat exchanger types. What is essential is a low pressure drop on the side of the cooling medium if a natural circulation is employed. To realize appropriate recirculation rates of the cooling medium in one example the liquid conveying height from the cooling reservoir may be configured having regard to the total pressure drop in the cooling circuit.

One option for achieving the desired subcooling could be an air cooler installed in the downpipe between the coolant reservoir and the inlet of the reactor cooling side. The cross sectional area of the heat exchanger for the coolant side is configured such that a low pressure drop is achieved.

To adapt the temperature upon entry of the cooling medium into the cooling side of the reactor a bypass around the heat exchanger for the subcooling may be used to reduce the flow through the heat exchanger, thus leading to a reduced subcooling. In the bypass in one example a control valve is installed to control the desired flow through the bypass/through the heat exchanger. A further valve could optionally be installed downstream of the heat exchanger to throttle the flow through the heat exchanger to a minimum if required. This would make it possible to completely bypass the heat exchanger. In one example the control valve is controlled by a temperature controller which controls the temperature of the cooling medium before entry into the cooling side of the reactor. In a further example the measured temperature of the reactor product gas on the process side at the reactor outlet could be used to control the bypass valve on the abovementioned temperature controller for the cooling directly or indirectly via a cascade.

When using an air cooler the cooling may also be controlled via the customary control devices (for example speed control, fan blade adjustment, shutter). However, the bypass solution would be suitable for all types of heat exchangers.

For improved heat integration it is useful according to one example to use a heat exchanger cooled with the cooling medium discharged from the reactor instead of an air cooler. This preheats the cooling medium while on the hot side of the heat exchanger the cooling medium of the reactor cooling circuit is cooled. Preheating makes it possible to recover more heat from the system and increase steam production in the steam drum.

Such a heat exchanger may be for example a double-tube heat exchanger, a multi-tube heat exchanger, a plate heat exchanger or another type of heat exchanger. In any case a countercurrent system between the hot and cold side is preferable to achieve optimal control and efficiency. In addition, the design of the heat exchanger must ensure a low pressure drop on the hot side connected to the coolant circuit of the reactor system. In one example the heat exchanger is integrated into the conduit between the coolant reservoir (steam drum) and the inlet of the reactor cooling side. In the case of an upright arrangement the flow on the hot side is from the upper end of the heat exchanger in a downward direction while the preheating of the cooling medium on the cold side is preferably from the lower inlet in an upward direction. In addition, in one example the design of the heat exchanger is such that each side of the heat exchanger uses only one pass and multiple passes are avoided to ensure good controllability of the heat transfer power.

The invention proposes a reactor for performing exothermic equilibrium reactions, in particular for performing methanol synthesis and/or ammonia synthesis by heterogeneously catalysed reaction of synthesis gas which makes it possible to influence and thus optimize the reaction conditions along the longitudinal coordinate of the reactor which, for example in the case of methanol synthesis, leads to a reduction in the recycle ratio to smaller values such as are known when using the reactors familiar from the prior art. Corresponding recycle conduits, circuit compressors etc. can therefore be made smaller or in some cases may even be entirely omitted. This reduces the corresponding capital costs.

Optimizing the reaction temperature profile along the longitudinal coordinate of the reactor further reduces the formation of undesired byproducts, thus affording a purer target product and reducing purification effort.

### Further Particular Embodiments of the Invention

In a second aspect of the invention the reactor according to the invention is characterized in that the first cooled section of the at least one tubular reactor or plate reactor comprises the last section of the catalyst fill in the flow direction of the input mixture stream and the product mixture stream. The first cooled section of the reactor coincides with the last section of the catalyst fill. Since coolant introduced into the interior space of this section has been subcooled relative to its boiling point with the coolant cooling apparatus said coolant is present in liquid form, thus allowing cooling temperatures below the boiling point to be established. The resulting temperature reduction in the catalyst bed thus makes it possible to shift the reaction equilibrium in this portion of the catalyst bed in the direction of the reaction products. This allows additional formation of reaction products even in the case where the reaction equilibrium has previously, i.e. in a first portion of the catalyst bed, already been established.

In a third aspect of the invention the reactor according to the invention is characterized in that the last section of the catalyst fill accounts for between 0% and 30% of the total length of the catalyst fill, preferably between 0% and 40% of the total length of the catalyst fill, most preferably between 0% and 50% of the total length of the catalyst fill. Studies have shown that corresponding sizing of the first cooled section of the reactor makes it possible to achieve particularly advantageous product yields. If, by contrast, the first cooled section of the reactor is made larger still, the second cooled section of the reactor may become too small, with the result that the reactant conversions/product yields achieved here are too small and too little reaction product is produced on balance over the entire reactor.

In a fourth aspect of the invention the reactor according to the invention is characterized in that the coolant cooling apparatus is adjustable such that the temperature of the coolant stream before entry into the interior space is at least 3° C., preferably at least 10° C., most preferably at least 20° C., below the boiling temperature of the coolant at the coolant pressure. Studies have shown that corresponding selection of the subcooling temperatures in the first cooled section of the reactor makes it possible to achieve particularly advantageous product yields.

In a fifth aspect of the invention the reactor according to the invention is characterized in that the reactor comprises a multiplicity of tubular reactors or plate reactors filled with catalyst fills which are arranged in the shell in parallel with respect to their longitudinal axis. Multiplication of the number of reactor tubes in the cooling shell results in multiplication of the product yield.

In a sixth aspect of the invention the reactor according to the invention is characterized in that the reactor is arranged upright with respect to its longitudinal axis so that the reactant inlet and the coolant outlet are located at the upper end of the reactor and the product outlet and the coolant inlet are located at the lower end of the reactor. This is especially advantageous since this has the result that the second cooled section is arranged at the top of the reactor. Since the coolant boils in this second cooled section of the reactor and is discharged from the interior space as vapour or as a biphasic flow the conveying of the coolant may be effected at least partially by natural circulation with the result that the corresponding conveying apparatuses, for example coolant pumps, may be made smaller or completely omitted.

A seventh aspect of the invention relates to the use of a reactor according to any of the abovementioned aspects for performing methanol synthesis and/or for ammonia synthesis.

In a ninth aspect of the invention the process according to the invention is characterized in that the first cooled section of the at least one tubular reactor or plate reactor comprises the last section of the catalyst fill in the flow direction of the input mixture stream and the product mixture stream. The advantages associated with this embodiment of the process correspond to those elucidated for the reactor according to the invention in connection with the second aspect of the invention.

In a tenth aspect of the invention the process according to the invention is characterized in that the last section of the catalyst fill accounts for between 0% and 30% of the total length of the catalyst fill, preferably between 0% and 40% of the total length of the catalyst fill, most preferably between 0% and 50% of the total length of the catalyst fill. The advantages associated with this embodiment of the process correspond to those elucidated for the reactor according to the invention in connection with the third aspect of the invention.

In an eleventh aspect of the invention the process according to the invention is characterized in that the coolant cooling apparatus is adjustable such that the temperature of the coolant stream before entry into the interior space is at least 3° C., preferably at least 10° C., most preferably at least 20° C., below the boiling temperature of the coolant at the coolant pressure. The advantages associated with this embodiment of the process correspond to those elucidated for the reactor according to the invention in connection with the fourth aspect of the invention.

In a twelfth aspect of the invention the process according to the invention is characterized in that the reactor comprises a multiplicity of tubular reactors or plate reactors filled with catalyst fills which are arranged in the shell in parallel with respect to their longitudinal axis. The advantages associated with this embodiment of the process correspond to those elucidated for the reactor according to the invention in connection with the fifth aspect of the invention.

In a thirteenth aspect of the invention the process according to the invention is characterized in that the reactor is arranged upright with respect to its longitudinal axis so that the reactant inlet and the coolant outlet are located at the upper end of the reactor and the product outlet and the coolant inlet are located at the lower end of the reactor. The advantages associated with this embodiment of the process correspond to those elucidated for the reactor according to the invention in connection with the sixth aspect of the invention.

In a fourteenth aspect of the invention the process according to the invention is characterized in that steps (2) to (5) are configured as follows:
(2) providing a gaseous input mixture stream containing hydrogen and carbon oxides as reactant components;
(3) introducing the gaseous input mixture stream via the reactant inlet into the at least one tubular reactor or plate reactor;
at least partially reacting the gaseous input mixture stream under methanol
(4) synthesis conditions to afford a fluid product mixture stream containing methanol as the product component and unconverted reactant components;
(5) discharging the fluid product mixture stream from the at least one tubular reactor or plate reactor via the product outlet.

This affords an advantageous process for methanol synthesis with the reactor according to the invention.

In a fifteenth aspect of the invention the process according to the invention is characterized in that steps (2) to (5) are configured as follows:
(2) providing a gaseous input mixture stream containing hydrogen and nitrogen as reactant components;
(3) introducing the gaseous input mixture stream via the reactant inlet into the at least one tubular reactor or plate reactor;
(4) at least partially reacting the gaseous input mixture stream under ammonia synthesis conditions to afford a gaseous product mixture stream containing ammonia as the product component and unconverted reactant components;
(5) discharging the gaseous product mixture stream from the at least one tubular reactor or plate reactor via the product outlet.

This affords an advantageous process for ammonia synthesis with the reactor according to the invention.

### BRIEF DESCRIPTION OF THE FIGURES

Developments, advantages and possible applications of the invention are also apparent from the following description of working and numerical examples and the drawings. The invention is formed by all of the features described and/or depicted, either on their own or in any combination, irrespective of the way they are combined in the claims or the dependency references therein.

In the figures:

FIG. 1 is a schematic diagram of a plant/a process comprising the reactor according to the invention;

FIG. 2 is an axial temperature profile in the reactor according to a first embodiment of the invention;

FIG. 3 is an axial temperature profile in the reactor according to a second embodiment of the invention;

FIG. 4 is an axial temperature profile in the reactor according to a third embodiment of the invention.

### DETAILED DESCRIPTION OF THE INVENTION

In the following, “not shown” is to be understood as meaning that an element in the figure under discussion is not graphically represented but nevertheless present in accordance with the description.

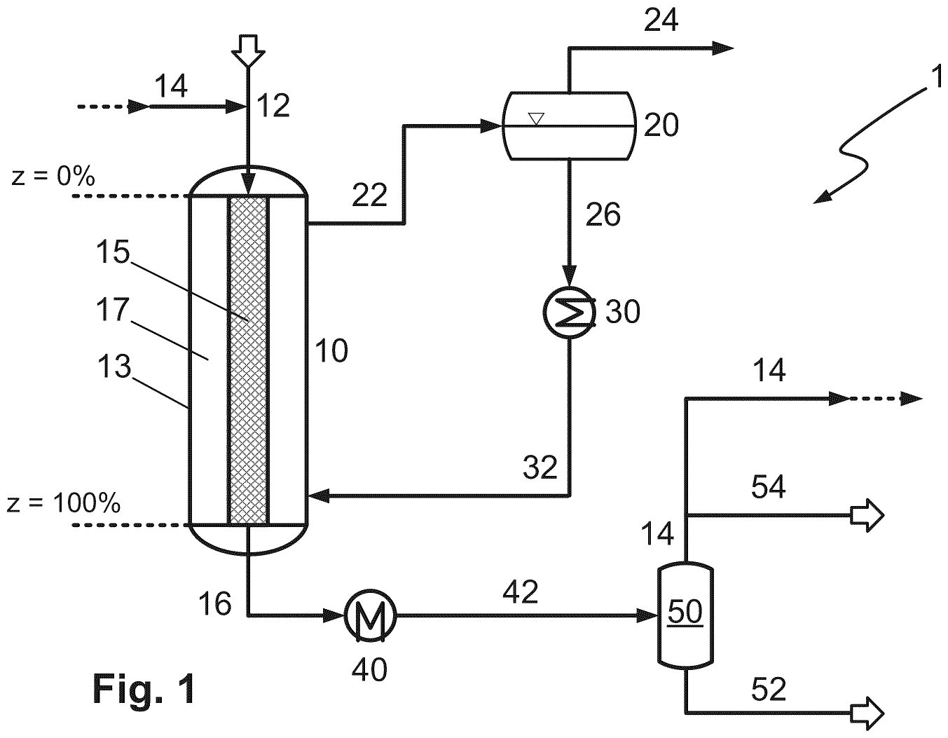

FIG. 1 shows an embodiment of a plant 1/a process 1 for methanol synthesis comprising a reactor 10 according to the invention.

A fresh synthesis gas stream (make-up gas stream) containing hydrogen and carbon oxides and inert components, for example methane, is introduced into the plant/into the process via a conduit 12 and introduced into a tubular reactor 15 arranged in the interior of a cylindrical shell 13 via a reactant inlet (not shown). Prior to this a recirculated gas stream containing unconverted synthesis gas constituents is supplied in a conduit 14 and likewise introduced into the tubular reactor 15 via conduit 12. Both the fresh synthesis gas and the recirculated gas are compressed to reaction pressure and conveyed using compressors (not shown) before introduction into the tubular reactor 15.

In the tubular reactor 15 a partial conversion of the synthesis gas components into methanol is effected over a methanol synthesis catalyst under methanol synthesis conditions, heat being liberated on account of the exothermic character of the reactions associated with methanol synthesis. The tubular reactor 15 is configured as a cylindrical single tube reactor and is filled with a catalyst active for methanol synthesis. The longitudinal coordinate z of the catalyst bed is indicated in FIG. 1. In the present example reactor 10 is in an upright arrangement.

Via a conduit 16 a product mixture stream containing methanol, unconverted synthesis gas constituents and inert components is discharged from the tubular reactor 15 and the reactor 10 via a product outlet (not shown), cooled to a temperature below its dew point using a cooler 40 and introduced into a gas-liquid phase separator 50 via a conduit 42.

In the gas-liquid phase separator 50 the product mixture stream is resolved into a liquid raw methanol stream which is discharged from the gas-liquid phase separator 50 via a conduit 52 and sent to a product workup (not shown). The largest portion of the unconverted synthesis gas is discharged from the gas-liquid phase separator 50 via conduit 14 and, after compression, sent back to the reactor 10 via conduit 12. A smaller portion of the synthesis gas is discharged from the plant/the process as a purge stream via conduit 14 and conduit 54 to prevent accumulation of inert components.

The tubular reactor 15 is cooled using water as coolant which is supplied as a subcooled coolant stream via a conduit 32 and introduced via a coolant inlet (not shown) into the interior space 17 which is arranged between the outer wall of the tubular reactor 15 and the inner wall of the shell 13 and in the present example has an annular shape.

When flowing through the interior space 17 in countercurrent to the gas flow of the reactant gases/product gases the coolant absorbs a portion of the reaction heat liberated by the methanol synthesis. Due to the upright arrangement of the reactor 10 the coolant flows through the interior space 17 from bottom to top. Due to the subcooling of the coolant effected by a cooler 30 the coolant remains liquid in a first cooled section of the tubular reactor, namely the last section of the catalyst bed having regard to the reactant stream/product stream between the longitudinal coordinate 100% and a value of the longitudinal coordinate z between 0% and 100%. Upon flowing further through the interior space 17 the coolant begins to boil and undergoes complete or partial evaporation. The cooling of the upper region of the tubular reactor 15 on a second cooled section of the tubular reactor is therefore effected by vaporous coolant or a biphasic flow of vapour and liquid. The second cooled section of the tubular reactor 15 therefore corresponds to the first section of the catalyst bed having regard to the reactant stream/product stream between the longitudinal coordinate 0% and a value of the longitudinal coordinate z between 0% and 100%.

The evaporated or partially evaporated coolant is discharged from the reactor 10 via a conduit 22 and introduced into a coolant reservoir 20 configured as a steam drum. A portion of the steam generated is sent to consumers via a conduit 24. The coolant thus withdrawn from the cooling circuit is compensated by supplying a corresponding water stream as a coolant stream to the steam drum via a conduit (not shown). Liquid, steam-saturated coolant is discharged from the steam drum 20 via a conduit 26 and supplied to the cooler 30. The cooler 30 effects a defined subcooling of the coolant which is then recycled to the reactor 10 via conduit 32.

### Numerical Examples for Methanol Synthesis

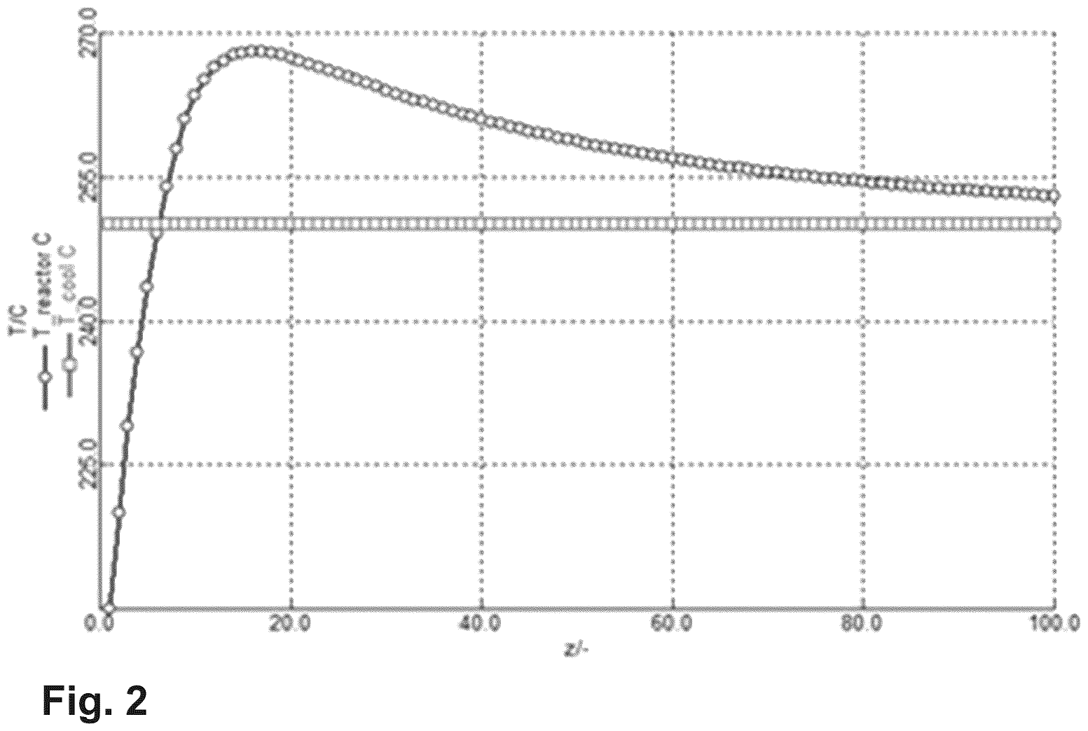

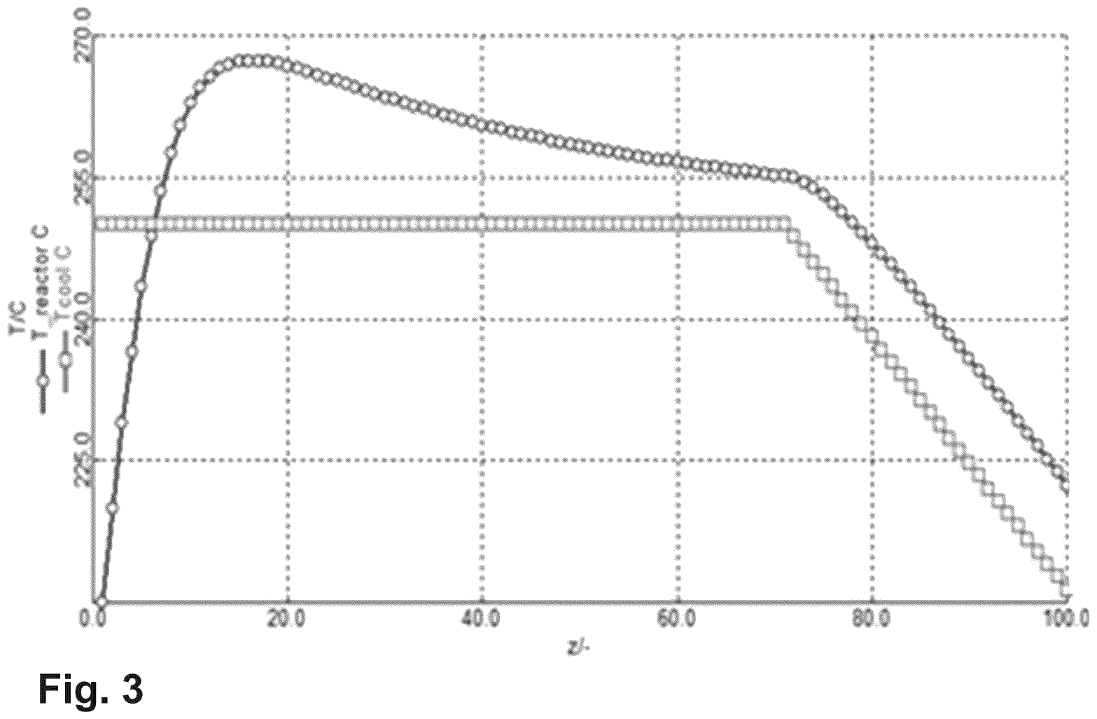

TABLE 1
Water-cooled reactor, 2000 TPD methanol
production. Reactor volume 88.8 m³

| | SOR (6 × 4) | | |
|---|---|---|---|
| | Comparative | | |
| | 100% | Inventive | Inventive |
| Make-up gas | constant | Last 30% | Last 30% |
| comprising | cooling | of cat. bed | of cat. bed |
| CO, CO₂, | temperature; | subcooled; | subcooled; |
| H₂; SN = 2.13 | FIG. 2 | FIG. 3 | FIG. 3 |
| MeOH/TPD | 2190 | 2206 | 2190 |
| X_CO2_pp | 0.32 | 0.35 | 0.33 |
| X_CO_pp | 0.78 | 0.83 | 0.83 |
| RR | 3.2 | 3.2 | 2.85 |

Key:
SN Stoichiometry number of methanol synthesis gas
SOR Start of Run (start of catalyst cycle)
EOR End of Run (end of catalyst cycle)
6 × 4, 3 × 3 Catalyst particle size
TPD (Metric) tonnes per day
X_CO2_pp CO2 conversion per reactor pass
X_CO_pp CO conversion per reactor pass
RR Recycle ratio of unconverted synthesis gas to reactor
LM Layer Management, layers of different catalysts

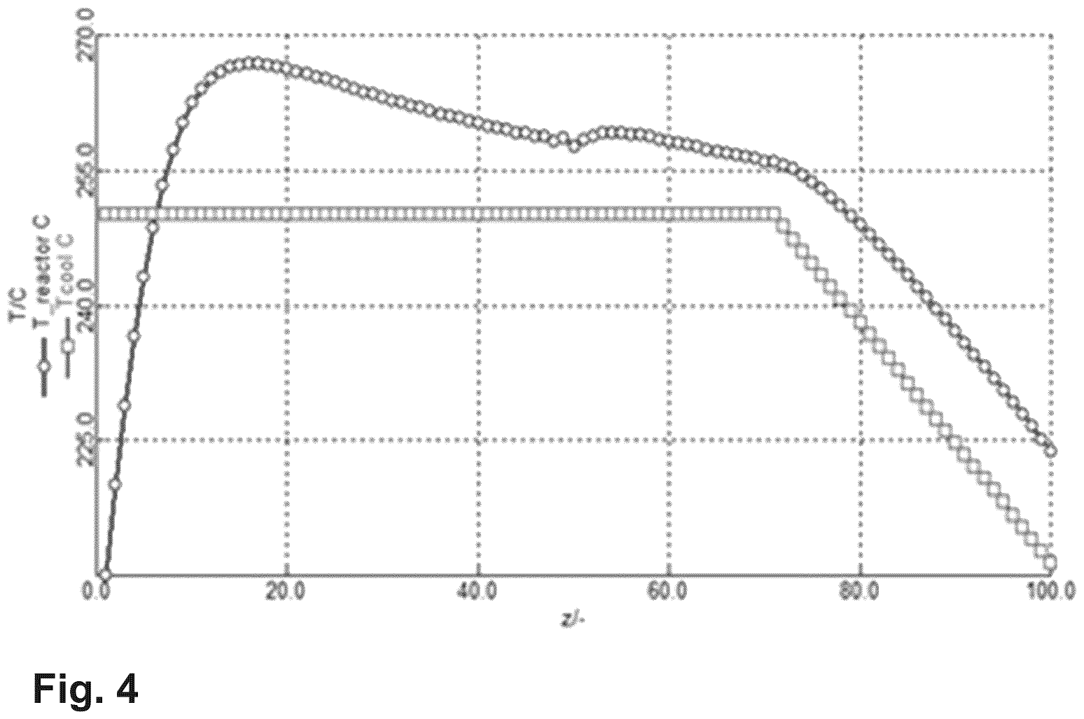

TABLE 2
Water-cooled reactor, 2000 TPD methanol,
layer management. Reactor volume 88.8 m³

| | SOR | | |
|---|---|---|---|
| | Comparative 1 | Comparative 2 | Inventive |
| | 100% | 100% constant | Last 30% |
| Make-up gas | constant | cooling | of cat. bed |
| comprising | cooling | temperature + | subcooled + LM |
| CO, CO₂, | temperature; | LM (50% 6 × 4, | (50% 6 × 4, 50% |
| H₂; SN = 2.13 | FIG. 2 | 50% 3 × 3) | 3 × 3); FIG. 4 |
| MeOH/TPD | 2190 | 2197 | 2213 |
| X_CO2_pp | 0.32 | 0.33 | 0.38 |
| X_CO_pp | 0.78 | 0.80 | 0.86 |
| RR | 3.2 | 3.2 | 3.2 |

As is apparent from the data summarized in table 1 subcooling of the last 30% of the catalyst bed increases methanol production from 2190 TPD to 2206 TPD (SOR). The invention alternatively makes it possible to reduce the recycle ratio from 3.2 to 2.85 while still retaining the same methanol production. The accompanying temperature profiles are shown in FIG. 2, wherein, as in FIG. 3 and FIG. 4, round symbols correspond to temperatures in the reactor (in the catalyst bed) and angular symbols correspond to temperatures of the coolant.

It is apparent from the data summarized in table 2 that the use of a catalyst layer management, i.e. the use of layers of different catalysts, here two catalysts for methanol synthesis of different particle size, together with the subcooling of the coolant according to the invention further increases production at constant recycle ratio. The associated temperature profiles are shown in FIG. 3.

TABLE 3
Water-cooled reactor, CO-rich synthesis gas, 2000 TPD methanol, layer management.

| | SOR | | | | | EOR | | | |
|---|---|---|---|---|---|---|---|---|---|
| Make-up gas | | | Inventive: | Inventive: | | | | | Inventive: |
| comprising | | Inventive: | LM | LM | | Inventive: | Inventive: | Inventive: | Cooling |
| CO, CO₂, H₂; | | Cooling | Cooling | Cooling | | Cooling | Cooling | Cooling | 40% + LM + |
| SN = 1.96 | Comparative | 40% | 40% | 40% + RR | Comparative | 40% | 40% + RR | 40% + LM | RR |
| MeOH/TPD | 2390 | 2406 | 2415 | 2390 | 2285 | 2302 | 2285 | 2348 | 2285 |
| X_CO2_pp | 0.075 | 0.077 | 0.08 | 0.09 | 0.061 | 0.056 | 0.060 | 0.062 | 0.068 |
| X_CO_pp | 0.55 | 0.605 | 0.64 | 0.75 | 0.38 | 0.41 | 0.50 | 0.47 | 0.65 |
| RR | 3.2 | 3.2 | 3.2 | 1.94 | 3.2 | 3.2 | 2.3 | 3.2 | 1.42 |

Reactor volume 88.8 m³

It is apparent from the data summarized in table 3 that the relationships elucidated in connection with table 1 and table 2 apply not only to SOR but also to EOR and performance of the process with Cl-rich synthesis gas. The associated temperature profiles are shown in FIG. 4.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing i.e. anything else may be additionally included and remain within the scope of “comprising.” “Comprising” is defined herein as necessarily encompassing the more limited transitional terms “consisting essentially of” and “consisting of”; “comprising” may therefore be replaced by “consisting essentially of” or “consisting of” and remain within the expressly defined scope of “comprising”.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

### LIST OF REFERENCE NUMERALS

[1] Plant/process
[10] Reactor
[12] Conduit
[13] Shell
[14] Conduit
[15] Tubular reactor
[16] Conduit
[17] Interior space
[20] Coolant reservoir (steam drum)
[22] Conduit
[24] Conduit
[26] Conduit
[30] Cooler (heat exchanger or air cooler)
[32] Conduit
[40] Cooler
[42] Conduit
[50] Gas-liquid phase separator
[52] Conduit
[54] Conduit

1. Reactor for performing exothermic equilibrium reactions where a fluid input mixture stream is at least partially reacted over a solid catalyst to afford a fluid product mixture stream, wherein the reactor comprises the following constituents in fluid connection with one another:
(a) at least one tubular reactor or plate reactor in which a catalyst is arranged as a fluid-permeable catalyst fill of solid catalyst particles, wherein the catalyst is active for performing the exothermic equilibrium reaction in a reaction temperature range at a reaction pressure;
(b) wherein the at least one tubular reactor or plate reactor bears the reaction pressure and at its one end comprises a reactant inlet for the fluid input mixture stream and at its other end comprises a product outlet for the fluid product mixture stream;
(c) a shell which surrounds the at least one tubular reactor or plate reactor and at its ends is fluid-tightly sealed with respect to the at least one tubular reactor or plate reactor, wherein the shell has a coolant inlet and a coolant outlet for a fluid coolant stream at opposite ends of the shell;
(d) wherein the interior space between the outer wall of the at least one tubular reactor or the outer walls of the at least one plate reactor and the inner wall of the shell is traversable by the coolant stream, wherein the coolant inlet and the coolant outlet are arranged such that the coolant stream passes through the reactor in countercurrent to the input mixture stream and the product mixture stream;
(e) a coolant for producing the coolant stream, wherein the coolant is selected such that it undergoes evaporation in the reaction temperature range at a specified coolant pressure;
(f) a pressure control apparatus for adjusting the coolant pressure in the interior space;
(g) a coolant cooling apparatus which makes it possible for the coolant stream to enter into the interior space in the liquid state via the coolant inlet and flow through the interior space initially as a liquid and thus cool a first cooled section of the at least one tubular reactor or plate reactor, then evaporate and in the vaporous state and/or as a biphasic stream of liquid and vaporous coolant cool the remaining, second cooled section of the at least one tubular reactor or plate reactor and then in the vaporous state and/or as a biphasic flow of liquid and vaporous coolant be discharged from the interior space via the coolant outlet.

2. Reactor according to claim 1, characterized in that the first cooled section of the at least one tubular reactor or plate reactor comprises the last section of the catalyst fill in the flow direction of the input mixture stream and the product mixture stream.

3. Reactor according to claim 1 or 2, characterized in that the last section of the catalyst fill accounts for between 0% and 30% of the total length of the catalyst fill, preferably between 0% and 40% of the total length of the catalyst fill, most preferably between 0% and 50% of the total length of the catalyst fill.

4. Reactor according to any of claims 1 to 3, characterized in that the coolant cooling apparatus is adjustable such that the temperature of the coolant stream before entry into the interior space is at least 3° C., preferably at least 10° C., most preferably at least 20° C., below the boiling temperature of the coolant at the coolant pressure.

5. Reactor according to any of claims 1 to 4, characterized in that the reactor comprises a multiplicity of tubular reactors or plate reactors filled with catalyst fills which are arranged in the shell in parallel with respect to their longitudinal axis.

6. Reactor according to any of claims 1 to 5, characterized in that the reactor is arranged upright with respect to its longitudinal axis so that the reactant inlet and the coolant outlet are located at the upper end of the reactor and the product outlet and the coolant inlet are located at the lower end of the reactor.

7. Use of a reactor according to any one of claims 1 to 6 for methanol synthesis and/or for ammonia synthesis.

8. Process for performing exothermic equilibrium reactions where a fluid input mixture stream is at least partially reacted over a solid catalyst to afford a fluid product mixture stream, wherein the process comprises the steps of:
(1) providing a reactor comprising:
(a) at least one tubular reactor or plate reactor in which a catalyst is arranged as a fluid-permeable catalyst fill of solid catalyst particles, wherein the catalyst is active for performing the exothermic equilibrium reaction in a reaction temperature range at a reaction pressure;
(b) wherein the tubular reactor or plate reactor bears the reaction pressure and at its one end comprises a reactant inlet for the fluid input mixture stream and at its other end comprises a product outlet for the fluid product mixture stream;
(c) a shell which surrounds the at least one tubular reactor or plate reactor and at its ends is fluid-tightly sealed with respect to the at least one tubular reactor or plate reactor, wherein the shell has a coolant inlet and a coolant outlet for a fluid coolant stream at opposite ends of the shell;
(d) wherein the interior space between the outer wall of the at least one tubular reactor or the outer walls of the at least one plate reactor and the inner wall of the shell is traversable by the coolant stream, wherein the coolant inlet and the coolant outlet are arranged such that the coolant stream passes through the reactor in countercurrent to the input mixture stream and the product mixture stream;
(e) a coolant for producing the coolant stream, wherein the coolant is selected such that it undergoes evaporation in the reaction temperature range at a specified coolant pressure;
(f) a pressure control apparatus for adjusting the coolant pressure in the interior space;
(g) a coolant cooling apparatus which makes it possible for the coolant stream to enter into the interior space in the liquid state via the coolant inlet and flow through the interior space initially as a liquid and thus cool a first cooled section of the at least one tubular reactor or plate reactor, then evaporate and in the vaporous state and/or as a biphasic stream of liquid and vaporous coolant cool the remaining, second cooled section of the at least one tubular reactor and then in the vaporous state and/or as a biphasic flow of liquid and vaporous coolant be discharged from the interior space via the coolant outlet;
(2) providing a fluid input mixture stream containing reactant components;
(3) introducing the fluid input mixture stream via the reactant inlet into the at least one tubular reactor or plate reactor;
(4) at least partially reacting the gaseous input mixture stream under conditions of exothermic equilibrium reaction to afford a fluid product mixture stream containing product components and unconverted reactant components;
(5) discharging the fluid product mixture stream from the at least one tubular reactor or plate reactor via the product outlet.

9. Process according to claim 8, characterized in that the first cooled section of the at least one tubular reactor or plate reactor comprises the last section of the catalyst fill in the flow direction of the input mixture stream and the product mixture stream.

10. Process according to claim 8 or 9, characterized in that the last section of the catalyst fill accounts for between 0% and 30% of the total length of the catalyst fill, preferably between 0% and 40% of the total length of the catalyst fill, most preferably between 0% and 50% of the total length of the catalyst fill.

11. Process according to any of claims 8 to 10, characterized in that the coolant cooling apparatus is adjustable such that the temperature of the coolant stream before entry into the interior space is at least 3° C., preferably at least 10° C., most preferably at least 20° C., below the boiling temperature of the coolant at the coolant pressure.

12. Process according to any of claims 8 to 11, characterized in that the reactor comprises a multiplicity of tubular reactors or plate reactors filled with catalyst fills which are arranged in the shell in parallel with respect to their longitudinal axis.

13. Process according to any of claims 8 to 12, characterized in that the reactor is arranged upright with respect to its longitudinal axis so that the reactant inlet and the coolant outlet are located at the upper end of the reactor and the product outlet and the coolant inlet are located at the lower end of the reactor.

14. Process according to any of claims 8 to 13, characterized in that steps (2) to (5) are configured as follows:
(2) providing a gaseous input mixture stream containing hydrogen and carbon oxides as reactant components;
(3) introducing the gaseous input mixture stream via the reactant inlet into the at least one tubular reactor or plate reactor;
(4) at least partially reacting the gaseous input mixture stream under methanol synthesis conditions to afford a fluid product mixture stream containing methanol as the product component and unconverted reactant components;
(5) discharging the fluid product mixture stream from the at least one tubular reactor or plate reactor via the product outlet.

15. Process according to any of claims 8 to 13, characterized in that steps (2) to (5) are configured as follows:
(2) providing a gaseous input mixture stream containing hydrogen and nitrogen as reactant components;
(3) introducing the gaseous input mixture stream via the reactant inlet into the at least one tubular reactor or plate reactor;
(4) at least partially reacting the gaseous input mixture stream under ammonia synthesis conditions to afford a gaseous product mixture stream containing ammonia as the product component and unconverted reactant components;
(5) discharging the gaseous product mixture stream from the at least one tubular reactor or plate reactor via the product outlet.
